(19) United States
(12) Patent Application Publication
(10) Pub. No.: US 2024/0079128 A1
(43) Pub. Date: Mar. 7, 2024

(54) SYSTEMS AND METHODS FOR PROVIDING DIGITAL HEALTHCARE SERVICES

(71) Applicant: Shanghai United Imaging Intelligence Co., Ltd., Shanghai (CN)

(72) Inventor: Terrence Chen, Lexington, MA (US)

(73) Assignee: Shanghai United Imaging Intelligence Co., Ltd., Shanghai (CN)

(21) Appl. No.: 17/901,349

(22) Filed: Sep. 1, 2022

(51) Int. Cl. G16H 40/67; G16H 30/40; G16H 40/20; G16H 50/20

(52) U.S. Cl. CPC G16H 40/67; G16H 30/40; G16H 40/20; G16H 50/20

(57) ABSTRACT

Traditional ways of seeking and receiving healthcare services are time-consuming and cumbersome. A digital healthcare service platform built on artificial intelligence (AI) technologies may improve the experience and efficiency associated with these services. The digital healthcare platform may use AI models trained for image classification and/or natural language processing to generate preliminary diagnoses for a care seeker based on images or descriptions provided by the care seeker. The digital healthcare platform may also use AI models to match service providers with the care seeker, and/or manage the logistical aspects of a service (e.g., coordinating activities, scheduling appointments, etc.) for the care seeker.

### BACKGROUND

Seeking healthcare services in a convention manner can be time-consuming and cumbersome as it may require searching for the right caregiver, making multiple appointments with various entities (e.g., doctors, labs, etc.), commuting to and from a medical facility, and receiving diagnoses and/or advice through in-person consultation. These activities not only consume resources, but also cause delays in providing a patient with the necessary attention and treatments. With the advancement of communication and computer technologies such as artificial intelligence (AI) related technologies, it is desirable to reform the manner in which healthcare services are provided to improve patient experience and the efficiency and quality of the healthcare services.

### SUMMARY

Described herein are systems, methods, and instrumentalities associated with providing and managing healthcare services using artificial intelligence (AI) based technologies. In accordance with one or more embodiments of the present disclosure, a digital healthcare platform may be provided, which may include an apparatus configured to receive a request for a medical service from a remote device (e.g., such as a computer or a smart phone), obtain one or more records associated with the medical service, and process the one or more records using at least a first AI model to generate a preliminary diagnosis for a person requesting the medical service. The one or more records may include pictures or images (e.g., medical scans) depicting a body area of the person or a description of symptoms experienced by the person, and the AI model may be trained to detect an abnormality in the pictures or images, or certain words in the description, link the abnormality or the words to a medical condition, and indicate the medical condition in the preliminary diagnosis.

The apparatus may transmit the preliminary diagnosis and/or a follow-up suggestion determined based on the preliminary diagnosis to the remote device, and may receive a response from the remote device indicating whether further medical assistance is needed by the requester. If the response indicates that further medical assistance is needed, the apparatus may further determine, using at least a second AI model, a list of providers capable of providing the further medical assistance and provide the list of providers to the remote device for the requester to select. The apparatus may additionally schedule an appointment with a provider selected by the requester on their behalf.

In examples, the one or more records used to generate the preliminary diagnosis may further include a medical history of the person requesting the medical service or biological information (e.g., age, gender, height, weight, etc.) of the person, and the first AI model may be trained to identify the person's medical condition further based on their medical history or the biological information. In some examples, the medical history and/or biological information may be provided by the person requesting the medical service. In other examples, the apparatus described herein may determine an identity of the person based on the request and may collect the medical history or the biological information of the person from sources of medical records based on the identity of the person.

In examples, the apparatus described herein may determine the list of providers that match the request by obtaining respective information regarding the list of providers and the person needing the further medical assistance, extracting, using the second AI model, respective attributes of the list of providers and the person from the obtained information, and matching the list of providers with the person based on the extracted attributes. In examples, the information regarding the list of providers may include one or more of respective services offered by the list of providers, respective availability of the list of providers, respective ratings of the list of providers, respective geographical locations of the list of providers, or respective types of insurance accepted by the list of providers. In examples, the information regarding the person needing the further medical assistance may include demographic information of the person, a desired time for the further medical assistance, a geographical location of the person, a type of insurance owned by the person, or the preliminary diagnosis generated by the first AI model.

### BRIEF DESCRIPTION OF THE DRAWINGS

A more detailed understanding of the examples disclosed herein may be had from the following description, given by way of example in conjunction with the accompanying drawing.

FIG. 1 is a simplified block diagram illustrating an example of providing healthcare services through a digital healthcare platform in accordance with one or more embodiments of the present disclosure.

FIG. 2A, FIG. 2B, and FIG. 2C are simplified block diagrams illustrating example AI models that may be used to enable the provisional of digital healthcare services in accordance with one or more embodiments of the present disclosure.

FIG. 3 is a flow diagram illustrating example operations that may be associated with training a neural network (e.g., an AI model implemented by the neural network) for performing a task described in one or more embodiments of the present disclosure.

FIG. 4 is a flow diagram illustrating example operations that may be associated with providing a digital healthcare service in accordance with one or more embodiments of the present disclosure.

FIG. 5 is a simplified block diagram illustrating example components of an apparatus that may be configured to perform the tasks described in one or more embodiments of the present disclosure.

### DETAILED DESCRIPTION

The present disclosure is illustrated by way of example, and not by way of limitation, in the figures of the accompanying drawings.

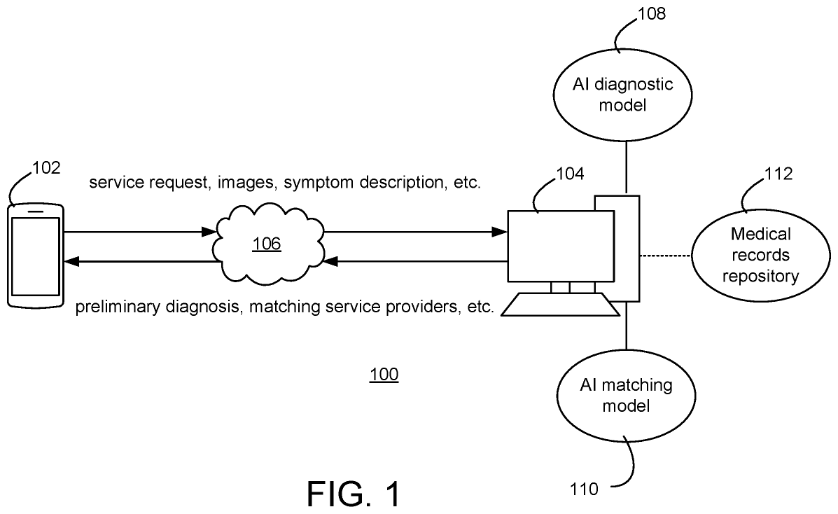

FIG. 1 illustrates an example of providing healthcare services through a digital healthcare platform 100 in accordance with one or more embodiments of the present disclosure. The digital healthcare platform 100 may include a user device 102 (e.g., one or more remote user devices) and a server device 104 (e.g., one or more server devices) that may be configured to communicate with the user device 102 via a communication network 106. In examples, the user device 102 may include a computer (e.g., a laptop or desktop computer), a smart device (e.g., a smart phone, a tablet, a wearable device such as a smart watch or activity tracker, etc.), a personal digital assistant (PDA), and/or another device capable of executing a set of instructions that may specify actions to be taken by that device. Similarly, the server device 104 may also include various types of computing devices such as desktop and/or laptop computers that may be programmed to process requests from the user device 102, execute one or more tasks to fulfill the requests, and provide responses to the user device 102 indicating a result of the task execution. While the server device 104 is shown as a single device in FIG. 1, those skilled in the art will understand that the server device 104 may include multiple computing devices (e.g., as parts of a cloud-based computing environment) configured to jointly fulfill the requests from the user device 102. Those skilled in the art will also understand that the communication network 106 may include a wired or a wireless network, or a combination thereof. For example, the communication network 106 may be established over a public network (e.g., the Internet), a private network (e.g., a local area network (LAN), a wide area network (WAN)), etc.), a wired network (e.g., an Ethernet network), a wireless network (e.g., an 802.11 network, a Wi-Fi network, etc.), a cellular network (e.g., a Long Term Evolution (LTE) or 5G network), a frame relay network, a virtual private network (VPN), a satellite network, and/or a telephone network. The communication network 106 may include one or more network access points. For example, the communication network 106 may include wired and/or wireless network access points such as base stations and/or internet exchange points through which one or more components of the digital healthcare platform 100 may be connected to exchange data and/or other information. Such exchange may utilize routers, hubs, switches, server computers, and/or any combination thereof.

The digital healthcare platform 100 may be used to service and/or connect at least two groups of users: care seekers (e.g., patients and/or their guardians or relatives) and caregivers (e.g., physicians, hospitals, nurses, physical therapists, etc.). The care seekers (also referred to as service recipients) may register on the digital platform and indicate their medical needs, while the caregivers (also referred to as service providers) may register on the digital platform and offer their services to the care seekers. Using artificial intelligence (AI) based technologies (e.g., artificial neural networks and/or machine-learning (ML) models implemented therein), the digital healthcare platform 100 may provide automated diagnoses and/or treatment advice to the care seekers, for example, based on information provided by and/or collected for the care seekers. Using the AI-based technologies, the digital healthcare platform 100 may also match additional needs of the care seekers (e.g., if the care seekers indicate such needs upon reviewing an automatically generated diagnosis) with the services offered by the caregivers, and provide (e.g., recommend) a list of caregivers for the care seekers to choose.

As shown in FIG. 1, AI-based technologies that may be used to perform the various functions of the digital healthcare platform 100 may include a first AI model 108 (e.g., a diagnostic model) trained for providing a preliminary diagnosis or advice to a care seeker and/or a second AI model 110 (e.g., a matching model) trained for matching additional needs of the care seeker to services offered by one or more caregivers. In an example scenario, the care seeker (e.g., such as a patient or a guardian or relative of the patient) may, upon registration with the digital healthcare platform 100, submit a request for a medical service from the user device 102 to the server device 104. The care seeker may indicate the desired medical service to the server device 104, for example, by submitting one or more records associated with the desired medical service to the serve device 104 (e.g., together with the request). These records may include, e.g., an image or picture that depicts a body area of the patient, a description of one or more symptoms (and duration of the symptoms) experienced by the patient, a medical history of the patient or the patient's family, biological information of the patient (e.g., age, gender, height, weight, body mass index (BMI), etc.), and/or the like. In some examples, the image or picture that depicts the body area of the patient may be taken by the patient (or the guardian or relative of the patient) and may show an abnormality in the body area (e.g., a tick bite, a mole, a suspicious lump, etc.). In other examples, the image or picture that depicts the body area of the patient may include a medical scan image of the patient such as a magnetic resonant imaging (MRI) image of the patient's heart or brain. Similarly, the description provided by the care seeker may, in some examples, include the care seeker's own words regarding the symptoms experienced by the patient and, in other examples, include a diagnosis received from a medical professional (e.g., if the care seeker wants to request a second opinion of the diagnosis from the digital healthcare platform).

In addition to (or in lieu of) receiving the one or more records from the user device 102, the server device 104 may (e.g., on its own) collect information about the patient needing the medical service. For example, based on the request submitted from the user device 102, the server device 104 may determine an identity of the patient (e.g., based on account information stored by the service device 104) and collect, from one or more sources, biological and/or medical information of the patient based on the patient's identity. As described herein, the biological information may include the age, gender, weight, height, and/or BMI of the patient, and the medical information may include a medical history of the patient and/or previous medical records of the patient (e.g., lab results, medical scans, prescriptions, etc.). The sources that may provide such information may include, for example, a medical records repository (e.g., 112 of FIG. 1, which may or may not be a part of the server device 104), a public website (not shown), a healthcare partner's databases (not shown), etc.

All or a subset of the records provided by the user device 102 or collected by the server device 104 may be used by the AI diagnostic model 108 to generate (e.g., without human intervention) a preliminary diagnosis for the care seeker. In examples, the AI diagnostic model 108 may include an image classification model that may be trained to receive as input an image of a body area of the care seeker and classify the image as indicating a certain medical condition or not indicating the medical condition (e.g., by outputting a probability score for the medical condition). For instance, if the image classification model determines, based on a picture of a wound suffered by the care seeker, that the care seeker only has a 10% chance of developing an infection from the wound, the image classification model may output a score of 0.1 to indicate the probability of infection. As another example, if the image classification model determines, based on a picture of the care seeker's retina, that there is a 80% chance that the care seekers has diabetes, the image classification model may output a score of 0.8 to indicate the probability of diabetes. The image classification model may be trained to generate these diagnoses (e.g., preliminary diagnoses) by identifying features associated with an abnormality (e.g., an infected wound, growth of abnormal blood vessels in the retina, etc.) in the input image and linking the abnormality to a corresponding medical condition (e.g., infection, diabetes, etc.). The training and implementation of such an image classification model will be described in greater detail below.

In examples, the AI diagnostic model 108 may include a semantics analysis model trained to receive as input a description of one or more symptoms experienced by the care seeks and determine, based on words contained in the description, that the symptoms may be associated with a certain medical condition. Similar to the image classification model described above, upon making the determination, the semantics analysis model may also output a probably score indicating the result of the determination. For instance, if the semantics analysis model detects words such as “dizziness,” “nausea,” and/or “vomiting,” the semantics analysis model may determine that the care seeker has a 60% chance of suffering from migraines and may output a score of 0.6 as the probability of migraines. As another example, if the semantics analysis model detects phases such as “sudden numbness or weakness in the face, arm, or leg,” the semantics analysis model may determine that the care seeker is at a 80% risk of having a stroke and may output a score of 0.8 to alert the care seeker about the risk. The semantics analysis model may be capable of generating these diagnoses (e.g., preliminary diagnoses) based on natural language processing skills acquired through training. The training and implementation of such an AI model will be described in greater detail below.

In examples, medical technology companies may register their solutions (e.g., AI-based solutions) with the digital healthcare platform 100 and make the solutions available to patients through the digital healthcare platform. Hence, in these examples, AI model 108 may include models developed and trained by these medical technology companies.

The preliminary diagnoses generated by AI model 108 may be used by the service device 104 to recommend additional actions to be taken by the care seeker. For example, based on a diagnosis of a potential heart condition, the server device 104 may recommend that the care seeker make an appointment with a cardiologist and the service device 104 may provide a list of cardiologists for the care seeker to choose. As another example, based on the detection of an abnormality in a chest X-ray of the care seeker, the service device 104 may recommend that a further scan of the area be conducted within three weeks. If a diagnosis indicates needs for emergency care, the server device 104 may urge the care seeker to visit an emergency room, and in some examples, with the care seeker's approval, the server device 104 may initiate contact with an ambulance service on behalf of the care seeker.

The server device 104 may transmit the preliminary diagnosis generated by AI model 108 and/or any follow-up suggestions to the care seeker (e.g., to the user device 102). In response, the care seeker may indicate to the server device 104 whether further medical assistance is desired by the care seeker. The care seeker may determine that further medical assistance is needed if the preliminary diagnosis indicates a serious medical condition or if the preliminary diagnosis is ambiguous. Upon receiving the indication that further medical assistance is desired by care seeker, the server device 104 may determine, using at least the second AI model 110, a list of providers that may be capable of providing the medical assistance and match one or more other conditions specified by the care seeker. In examples, the second AI model 110 may include a regression model trained to regress multiple pieces of input information related to care seekers and service providers to respective matching scores (e.g., between 0.0 to 1.0) indicating the likelihood that a care seeker may choose a certain service provider for a desired medical service. The second AI model may learn to solve such a high-dimension regression problem based on training and/or past user selections made on the digital healthcare system 100. So, the more the digital healthcare system 100 is used, the more accurate the matching score generated by the second AI model may become.

The input information that may be used to solve the high-dimension regression problem described above may include care seeker information such as the type(s) of medical assistance needed (e.g., AI-based, virtual, physical, etc.), the level of expertise or experience expected from a provider, preferred locations and/or times, type(s) of insurance owned, and/or the like that may be provided by the care seeker (e.g., at the time of registration or together with a specific request) or determined by the server device 104/user device 102 (e.g., the location of the care seeker may be automatically determined based on a GPS location and/or an IP address of the user device 102).

The input information used to solve the high-dimension regression problem described above may also include provider information such as the specialty of the provider (e.g., cardiology, dermatology, etc.), type(s) of medical services offered by the provider (e.g., AI-based, virtual, physical, etc.), the level of expertise or experience of the provider, available locations and/or times, type(s) of insurance accepted, and/or the like that may be entered by the provider (e.g., upon registration with the digital healthcare system 100). In examples, the provider information may also include information gathered by the digital healthcare system 100 from other sources including, for example, public reviews of the provider, ratings of the provider, etc. In examples, the preliminary diagnosis generated by AI model 108 may also be used as an additional input to solve the regression problem.

In examples, the matching scores generated by the second AI model may be used to filter and/or sort the providers recommended to the care seeker. For example, the service device 104 may decide to recommend only those providers having a matching score above a certain threshold to the care seeker. In other examples, alternative and/or additional attributes may be used to filter and/or sort the providers. For instance, the providers may be filter and/or sorted based on a distance of the providers from the care seeker (e.g., only those providers located within 10 miles of the care seeker may be listed and further sorted based on the distance).

Upon determining the list of matching providers using the second AI model 110, the service device 104 may provide the list to the care seeker (e.g., to the user device 102). The service device 104 may additionally indicate scheduling/availability information of the list of providers to the care seeker. If the care seeker selects one of the providers from the list and indicates the selection to the server device 104, the server device 104 may further schedule an appointment with the selected provider on behalf of the care seeker, and send a confirmation and/or reminder of the appointment to the care seeker. In examples, after completing an appointment, the care seeker may indicate on the digital healthcare system 100 that the appointment has been completed, and the server device may determine and/or trigger a next step for the care seeker. Hence, the digital healthcare platform 100 may be used to optimize the workflow of patient care, e.g., within the same integrated delivery network (IDN) or hospital network, or across multiple IDNs or hospital networks. In some examples, the digital healthcare platform 100 may also allow doctors to provide virtual medical services such as virtual surgeries during which multiple surgeons may remotely visualize a patient and provide opinions and guidance on a surgical operation. In some examples, the digital healthcare platform 100 may offer multiple AI-based services (e.g., AI models for automated diagnoses). Based on user request and/or data, if a corresponding AI model is available on the digital healthcare platform 100, the user request may be processed automatically and a diagnosis (or prescription) may be made available within a short period of time.

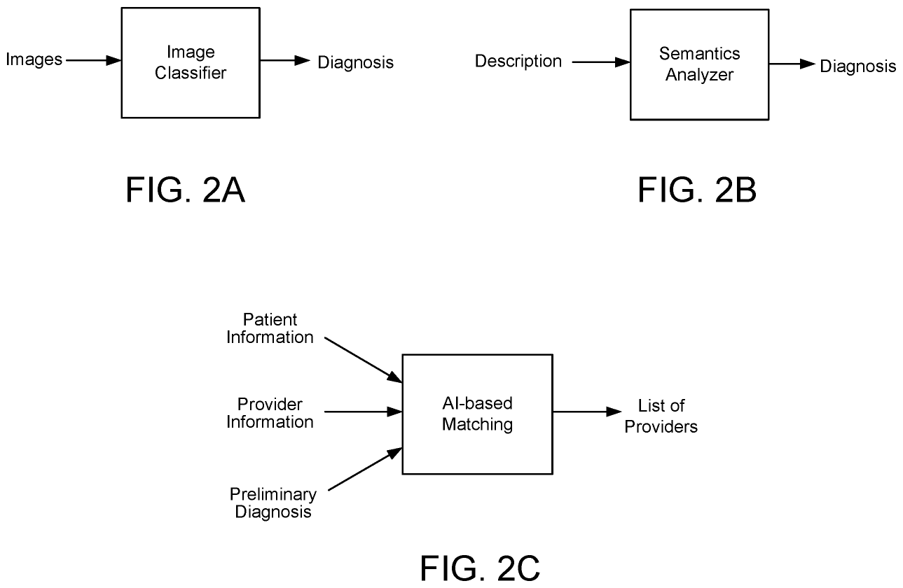

FIGS. 2A-2C illustrate examples of AI models that may be used to enable the provisional of digital healthcare services as described herein. FIG. 2A shows an example of an AI model that may be trained to operate as an image classifier (e.g., all or a part of the AI diagnostic model 108 in FIG. 1) to detect an abnormality in an image of a person. As described herein, the abnormality may be a tick bite, a mole, a suspicious lump or mass, etc., that may be suspicious of being linked to a certain medical condition, and the image of the person may include a picture taken and uploaded by a patient, a medical scan image provided by the patient or retrieved by a digital healthcare platform as described herein, etc. The image classifier model may be learned and/or implemented using an artificial neural network (ANN) such as a convolutional neural network (e.g., the AI models described herein may refer to the structure and/or parameters of the respective neural networks used to learn and/or implement the AI models). In examples, the ANN may include a plurality of layers such as one or more convolution layers, one or more pooling layers, and/or one or more fully connected layers. Each of the convolution layers may include a plurality of convolution kernels or filters configured to extract features from an input image. The convolution operations may be followed by batch normalization and/or linear (or non-linear) activation, and the features extracted by the convolution layers may be down-sampled through the pooling layers and/or the fully connected layers to reduce the redundancy and/or dimension of the features, so as to obtain a representation of the down-sampled features (e.g., in the form of a feature vector or feature map). In examples, the ANN may further include one or more un-pooling layers and one or more transposed convolution layers that may be configured to up-sample and de-convolve the features extracted through the operations described above. As a result of the up-sampling and de-convolution, a dense feature representation (e.g., a dense feature map) of the input image may be derived, and the ANN may be trained (e.g., parameters of the ANN may be adjusted) to predict the presence or non-presence of a target object (e.g., the abnormality described herein) in the input image based on the feature representation.

FIG. 2B shows an example of an AI model that may be trained to operate as a semantics analyzer (e.g., all or a part of the AI diagnostic model 108 in FIG. 1) to identify a potential medical issue based on a textual description provided by a care seeker that may describe symptoms experienced by the care seeker. The textual description may be provided by the care seeker using one or more controls on a user interface. For example, one or more check boxes may be provided for the care seeker to indicate whether he/she is experiencing some common medical conditions. As another example, a text field may be provided for the care seeker to describe his/her symptoms or conditions. The semantics analyzer may, through training, acquire natural language processing skills, with which it may automatically recognize certain texts contained in the description as being indicative of a potential medical condition. For example, the semantics analyzer may be able to parse descriptions or textual medical records (e.g., narratives, diagnoses, prescriptions, etc.), and link words such as “itchy throat,” “fever,” “coughing,” and/or “loss of smell” to “Covid 19,” link words such as “shortness of breath,” “sweating,” and/or “chest pain” to “heart attack,” etc. The semantics analyzer may be implemented using various neural network structures including, for example, the convolutional neural network (CNN) described herein, a recurrent neural network (RNN) (e.g., including a recursive neural network), a long-short term memory (LSTM) neural network, a graph neural network (GNN), and/or the like. Using a CNN as an example, such a network may include an input layer configured to tokenize an textual input and feed the tokenized texts to a plurality of convolutional layers to learn a hierarchy of representations and different levels of abstractions of the textual input, e.g., in the form of one or more encoded feature vectors or feature maps. These feature maps may then be pooled with a max operation to reduce the dimensionality of the representation, before the representation is provided to an output layer (e.g., one or more fully-connected layers) to predict a meaning of the input texts (e.g., classify the texts as having a certain meaning).

FIG. 2C shows an example of an AI model (e.g., the AI-based matching model 110 in FIG. 1) that may be trained to match care seekers (e.g., patients) with caregivers (e.g., doctors, hospitals, etc.) based on a plurality of criteria or conditions. In examples, such an AI model may be implemented using a regression neural network trained to predict an output as a function of multiple pieces of input information (e.g., all or a subset of the input information). For instance, the output of the AI model may include a matching score (e.g., having a value between 0.0 to 1.0) indicating the likelihood that a care seeker may choose a certain service provider for a desired medical service, and the input to the AI model may include patient information, provider information, and/or a preliminary diagnosis for the patient (e.g., the preliminary diagnosis generated in accordance with FIGS. 2A and 2B). The patient information that may be used to regress the matching score may include, for example, the type(s) of medical assistance (e.g., physical, virtual, hybrid, etc.) sought by a care seeker, the level of expertise or experience expected from a provider, preferred locations and/or times, type(s) of insurance owned, and/or the like. The provider information that may be used to regress the matching score may include, for example, the specialty of the provider (e.g., cardiology, dermatology, etc.), the type(s) of medical services offered by the provider (e.g., virtual, physical, hybrid, etc.), the level of expertise or experience of the provider, available locations and/or times, type(s) of insurance accepted, and/or the like.

In examples, the regression neural network used to implement the AI matching model may be a feedforward, fully connected neural network comprising an input layer, one or more fully connected layers, and/or an output layer. A first fully connected layer of the neural network may have a connection from the network input (e.g., predictor data comprising patient and provider attributes), and each subsequent layer may have a connection from the previous layer. Each fully connected layer may multiply its input by a weight matrix (e.g., kernel or filter weights) and/or add a bias vector to the resulting product. An activation function (e.g., a rectified linear unit (ReLU) activation function) may follow each fully connected layer (e.g., excluding the last), and the final fully connected layer may produce the network's output such as the matching score described herein. The weights of the regression neural network (e.g., parameters of the AI matching models) may be optimized using a loss function such as a mean squared error (MSE) loss function that may indicate a difference between a predicted score and a ground truth score.

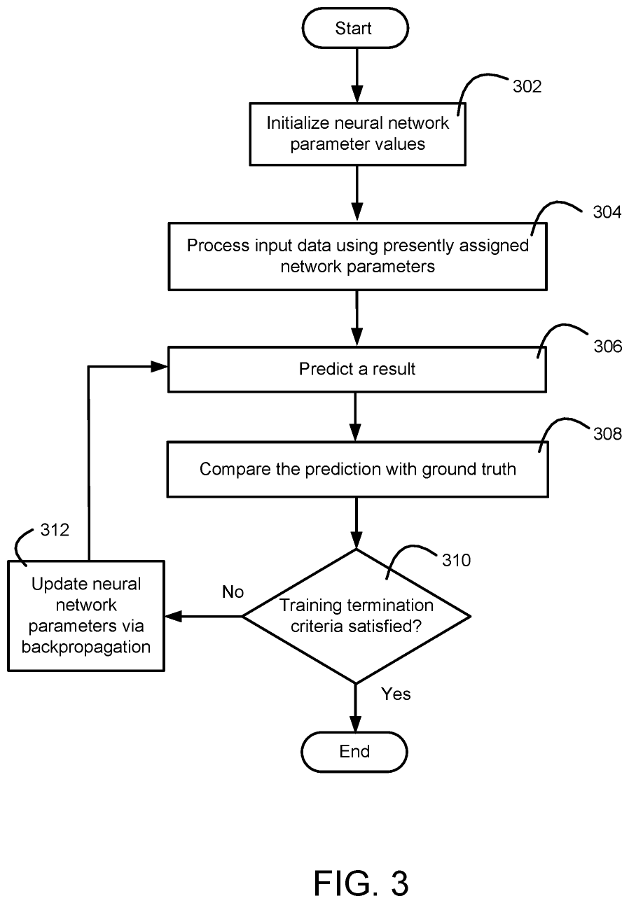

FIG. 3 illustrates an example procedure for training a neural network (e.g., an AI model implemented by the neural network) to perform one or more of the tasks described herein. As shown, the training process may include initializing the operating parameters of the neural network (e.g., weights associated with various layers of the neural network) at 302, for example, by sampling from a probability distribution or by copying the parameters of another neural network having a similar structure. The training process may further include processing an input (e.g., a picture, a medical scan image, a description of medical issues, regression predictor variables, etc.) using presently assigned parameters of the neural network at 304, and making a prediction for a desired result (e.g., a feature vector, a classification label, a matching score, etc.) at 306. The prediction result may then be compared to a ground truth at 308 to calculate a loss associated with the prediction based on a loss function such as an MSE, an L1 norm, an L2 norm, etc. The calculated loss may be used to determine, at 310, whether one or more training termination criteria are satisfied. For example, the training termination criteria may be determined to be satisfied if the loss is below a threshold value or if the change in the loss between two training iterations falls below a threshold value. If the determination at 310 is that the termination criteria are satisfied, the training may end; otherwise, the presently assigned network parameters may be adjusted at 312, for example, by backpropagating a gradient descent of the loss function through the network before the training returns to 306.

For simplicity of explanation, the training operations are depicted in FIG. 3 and described herein with a specific order. It should be appreciated, however, that the training operations may occur in various orders, concurrently, and/or with other operations not presented or described herein. Furthermore, it should be noted that not all operations that may be included in the training procedure are depicted and described herein, and not all illustrated operations are required to be performed.

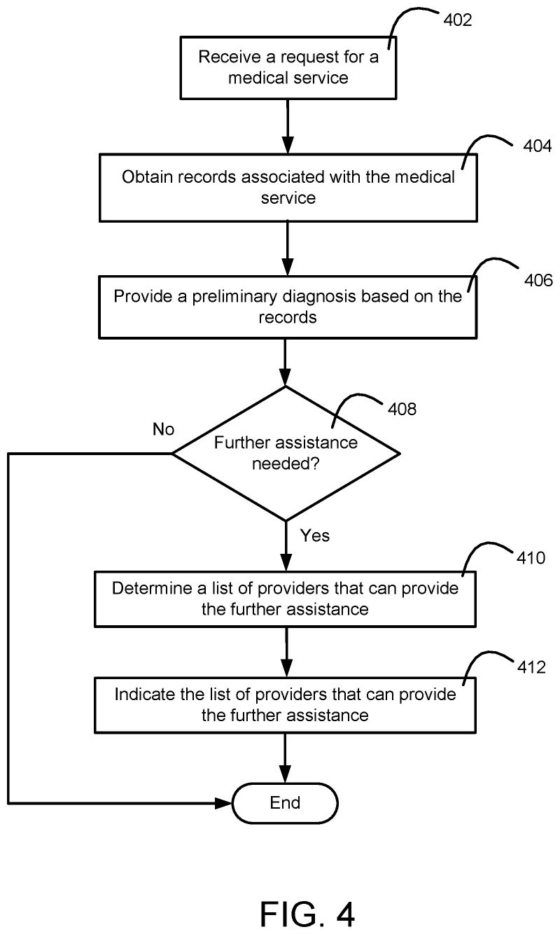

FIG. 4 illustrates example operations that may be associated with providing digital healthcare services in accordance with one or more embodiments of the present disclosure. As described herein, these operations may be performed by a server device (e.g., the server device 104 in FIG. 1) and/or a user device (e.g., the user device 102 in FIG. 1) in response to a request submitted by a care seeker (e.g., a patient). As show, the operations may include receiving a request for a medical service at 402, and obtaining one or more records associated with the medical service at 404. The request may be submitted by the care seeker, for example, via a software application (e.g., a desktop application or a mobile app) installed on a device of the care seeker (e.g., a desktop computer, a mobile device, etc.), and the one or more records obtained may include records (e.g., images, narratives, etc.) provided by the care seeker or retrieved by the server/user device. At 406, the one or more records may be processed using at least a first artificial intelligence (AI) model (e.g., an image classifier or a semantics analyzer) to generate a preliminary diagnosis for the care seeker. The preliminary diagnosis may be sent to the care seeker and a determination may be made at 408 (e.g., based on a response from the care seeker) regarding whether further medical assistance is needed. If the determination at 408 is that no further medical assistance is needed, the example operations may end. Otherwise, the example operations may further include determining, using at least a second AI model (e.g., a provider matching model), a list of providers that may be capable of providing the further medical assistance at 410 and indicate the list of providers to the care seeker at 412.

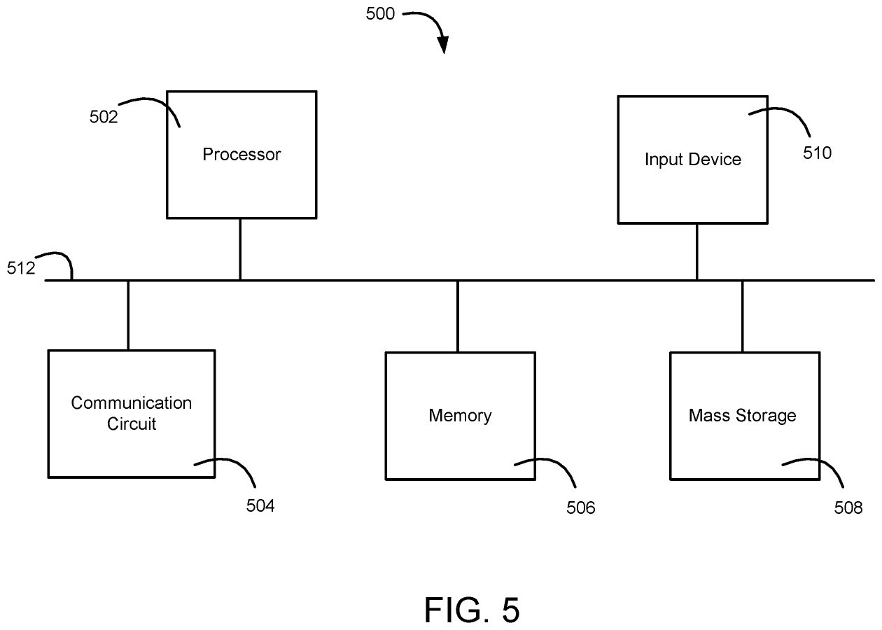

The systems, methods, and/or instrumentalities described herein may be implemented using one or more processors, one or more storage devices, and/or other suitable accessory devices such as display devices, communication devices, input/output devices, etc. FIG. 5 illustrates an example apparatus 500 that may be configured to perform the tasks described herein. As shown, apparatus 500 may include a processor (e.g., one or more processors) 502, which may be a central processing unit (CPU), a graphics processing unit (GPU), a microcontroller, a reduced instruction set computer (RISC) processor, application specific integrated circuits (ASICs), an application-specific instruction-set processor (ASIP), a physics processing unit (PPU), a digital signal processor (DSP), a field programmable gate array (FPGA), or any other circuit or processor capable of executing the functions described herein. Apparatus 500 may further include a communication circuit 504, a memory 506, a mass storage device 508, an input device 510, and/or a communication link 512 (e.g., a communication bus) over which the one or more components shown in the figure may exchange information.

Communication circuit 504 may be configured to transmit and receive information utilizing one or more communication protocols (e.g., TCP/IP) and one or more communication networks including a local area network (LAN), a wide area network (WAN), the Internet, a wireless data network (e.g., a Wi-Fi, 3G, 4G/LTE, or 5G network). Memory 506 may include a storage medium (e.g., a non-transitory storage medium) configured to store machine-readable instructions that, when executed, cause processor 502 to perform one or more of the functions described herein. Examples of the machine-readable medium may include volatile or non-volatile memory including but not limited to semiconductor memory (e.g., electrically programmable read-only memory (EPROM), electrically erasable programmable read-only memory (EEPROM)), flash memory, and/or the like. Mass storage device 508 may include one or more magnetic disks such as one or more internal hard disks, one or more removable disks, one or more magneto-optical disks, one or more CD-ROM or DVD-ROM disks, etc., on which instructions and/or data may be stored to facilitate the operation of processor 502. Input device 510 may include a keyboard, a mouse, a voice-controlled input device, a touch sensitive input device (e.g., a touch screen), and/or the like for receiving user inputs to apparatus 500.

It should be noted that apparatus 500 may operate as a standalone device or may be connected (e.g., networked or clustered) with other computation devices to perform the tasks described herein. And even though only one instance of each component is shown in FIG. 5, a skilled person in the art will understand that apparatus 500 may include multiple instances of one or more of the components shown in the figure.

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure. In addition, unless specifically stated otherwise, discussions utilizing terms such as “analyzing,” “determining,” “enabling,” “identifying,” “modifying” or the like, refer to the actions and processes of a computer system, or similar electronic computing device, that manipulates and transforms data represented as physical (e.g., electronic) quantities within the computer system's registers and memories into other data represented as physical quantities within the computer system memories or other such information storage, transmission or display devices.

It is to be understood that the above description is intended to be illustrative, and not restrictive. Many other implementations will be apparent to those of skill in the art upon reading and understanding the above description. The scope of the disclosure should, therefore, be determined with reference to the appended claims, along with the full scope of equivalents to which such claims are entitled.

What is claimed is:

1. An apparatus, comprising:
one or more processors configured to:
receive a request for a medical service from a remote device;
obtain one or more records associated with the medical service;
process the one or more records using at least a first artificial intelligence (AI) model to generate a preliminary diagnosis;
transmit the preliminary diagnosis to the remote device and receive a response indicating whether further medical assistance is needed; and
based on a determination that the response indicates that further medical assistance is needed:
determine, using at least a second AI model, a list of providers capable of providing the further medical assistance; and
provide the list of providers to the remote device.

2. The apparatus of claim 1, wherein the one or more records include an image that depicts a body area of a person or a description of a symptom experienced by the person, wherein the first AI model is trained to automatically identify a medical condition of the person based on the image or the description, and wherein the preliminary diagnosis indicates the automatically identified medical issue.

3. The apparatus of claim 2, wherein the image shows an abnormality in the body area of the person, and the first AI model is trained to recognize the abnormality based on features of the image that are associated with the abnormality, the first AI model further trained to link the abnormality with the medical condition.

4. The apparatus of claim 2, wherein the description of the symptom includes one or more words, and the first AI model is trained to recognize that the one or more words are associated with the medical condition.

5. The apparatus of claim 2, wherein the image or description is received from the remote device.

6. The apparatus of claim 2, wherein the one or more records further include a medical history of the person, a medical scan image of the person, or biological information of the person, and wherein the first AI model is trained to identify the medical condition further based on the medical history, the medical scan image, or the biological information of the person.

7. The apparatus of claim 6, wherein the one or more processors are further configured to determine an identity of the person based on the request and collect, from one or more sources, the medical history, the medical scan image, or the biological information of the person based on the identity of the person.

8. The apparatus of claim 6, wherein the one or more processors are further configured to receive the medical history, the medical scan image, or the biological information of the person from the remote device.

9. The apparatus of claim 1, wherein the one or more processors being configured to determine the list of providers capable of providing the further medical assistance comprises the one or more processors being configured to:
obtain respective information regarding the list of providers and a person needing the further medical assistance;
extract, using the second AI model, respective attributes of the list of providers and the person from the obtained information; and
match, using the second AI model, the list of providers with the person based on the extracted attributes.

10. The apparatus of claim 9, wherein the information regarding the list of providers indicates one or more of respective services offered by the list of providers, respective availability of the list of providers, respective ratings of the list of providers, respective geographical locations of the list of providers, or respective types of insurance accepted by the list of providers.

11. The apparatus of claim 9, wherein the information regarding the person needing the further medical assistance includes demographic information of the person, a desired time for the further medical assistance, a geographical location of the person, a type of insurance owned by the person, or the preliminary diagnosis generated by the first AI model.

12. The apparatus of claim 1, wherein the one or more processors are further configured to determine, based on the preliminary diagnosis generated by the first AI model, a next step to be taken and indicate the next step to the remoted device.

13. A method of providing healthcare services, the method comprising:
receiving a request for a medical service from a remote device;
obtaining one or more records associated with the medical service;
processing the one or more records using at least a first artificial intelligence (AI) model to generate a preliminary diagnosis;
transmitting the preliminary diagnosis to the remote device and receiving a response indicating whether further medical assistance is needed; and
based on a determination that the response indicates that further medical assistance is needed:
determining, using at least a second AI model, a list of providers capable of providing the further medical assistance; and
providing the list of providers to the remote device.

14. The method of claim 13, wherein the one or more records include an image that depicts a body area of a person or a description of a symptom experienced by the person, wherein the first AI model is trained to automatically identify a medical condition of the person based on the image or the description, and wherein the preliminary diagnosis indicates the automatically identified medical issue.

15. The method of claim 14, wherein the image shows an abnormality in the body area of the person, and the first AI model is trained to recognize the abnormality based on features of the image that are associated with the abnormality, the first AI model further trained to link the abnormality with the medical condition.

16. The method of claim 14, wherein the description of the symptom includes one or more words, and the first AI model is trained to recognize that the one or more words are associated with the medical condition.

17. The method of claim 14, wherein the one or more records further include a medical history of the person, a medical scan image of the person, or biological information of the person, and wherein the first AI model is trained to identify the medical condition further based on the medical history, the medical scan image, or the biological information of the person.

18. The method of claim 12, wherein determining the list of providers capable of providing the further medical assistance comprises:
obtaining respective information regarding the list of providers and a person needing the further medical assistance;
extracting, using the second AI model, respective attributes of the list of providers and the person from the obtained information; and
matching, using the second AI model, the list of providers with the person based on the extracted attributes.

19. The method of claim 18, wherein the information regarding the list of providers indicates one or more of respective services offered by the list of providers, respective availability of the list of providers, respective ratings of the list of providers, respective geographical locations of the list of providers, or respective types of insurance accepted by the list of providers, and wherein the information regarding the person includes demographic information of the person, a desired time for the further medical assistance, a geographical location of the person, a type of insurance owned by the person, or the preliminary diagnosis generated by the first AI model.

20. An apparatus, comprising:
one or more processors configured to:
receive a request for a medical service from a person;
obtain one or more records associated with the medical service;
process the one or more records using at least a first artificial intelligence (AI) model to generate a preliminary diagnosis;
present the preliminary diagnosis to the person and receive a response indicating whether further medical assistance is needed; and
based on a determination that the response indicates that further medical assistance is needed:
determine, using at least a second AI model, a list of providers capable of providing the further medical assistance; and
present the list of providers to the person
